(19) United States
(12) Patent Application Publication
(10) Pub. No.: US 2003/0091974 A1
(43) Pub. Date: May 15, 2003

(54) Method for screening compounds capable of depleting mast cells

(76) Inventors: Alain Moussy, Paris (FR); Jean-Pierre Kinet, Lexington, MA (US)

Correspondence Address: FOLEY AND LARDNER, SUITE 500, 3000 K STREET NW, WASHINGTON, DC, 20007, US

(21) Appl. No.: 10/022,842

(22) Filed: Dec. 20, 2001

Related U.S. Application Data
(60) Provisional application No. 60/301,404, filed on Jun. 29, 2001.
(60) Provisional application No. 60/301,405, filed on Jun. 29, 2001.
(60) Provisional application No. 60/301,406, filed on Jun. 29, 2001.
(60) Provisional application No. 60/301,407, filed on Jun. 29, 2001.
(60) Provisional application No. 60/301,408, filed on Jun. 29, 2001.
(60) Provisional application No. 60/301,409, filed on Jun. 29, 2001.
(60) Provisional application No. 60/301,410, filed on Jun. 29, 2001.
(60) Provisional application No. 60/301,411, filed on Jun. 29, 2001.
(60) Provisional application No. 60/323,313, filed on Sep. 20, 2001.
(60) Provisional application No. 60/323,314, filed on Sep. 20, 2001.
(60) Provisional application No. 60/323,315, filed on Sep. 20, 2001.

(51) Int. Cl. C12Q 1/00; G01N 33/567

(52) U.S. Cl. 435/4; 435/7.21

(57) ABSTRACT

A method of identifying compounds that deplete mast cells without depleting other related cells is described. The invention further relates to compounds identified by the screening method and their uses in treating diseases.

The present invention relates to a screening method allowing the identification and selection of compounds capable of depleting mast cells, wherein said compounds do not show significant toxicity for other hematopoeitic cells that are not mast cells or related cells or cell lines, such as SCF independent expanded human normal CD34+ cells.

Mast cells (MC) are tissue elements derived from a particular subset of hematopoietic stem cells that express CD34, c-kit and CD13 antigens (Kirshenbaum et al, Blood. 94: 2333-2342, 1999 and Ishizaka et al, Curr Opin Immunol. 5: 937-43, 1993). Immature MC progenitors circulate in the bloodstream and differentiate in tissues. These differentiation and proliferation processes are under the influence of cytokines, one of utmost importance being Stem Cell Factor (SCF), whose receptor is c-kit.

Mast cells are characterized by their heterogeneity, not only regarding tissue location and structure but also at the functional and histochemical levels (Aldenborg and Enerback., Histochem. J. 26: 587-96, 1994; Bradding et al. J Immunol. 155: 297-307, 1995; Irani et al, J Immunol. 147: 247-53, 1991; Miller et al, Curr Opin Immunol. 1: 637-42, 1989 and Welle et al, J Leukoc Biol. 61: 233-45, 1997). Indeed, at least three different subtypes of mast cells exist in humans, that differ by their morphological appearance, their tissue location, their biochemical content and their reactivity towards various compounds. These three different subtypes of mast cells are distinguished on the basis of their content of neutral proteases. Mast cells containing only tryptase (T) are termed MCT, while MC containing tryptase and chymase (C) are known as MCTC. Additionally, a minor population of mast cells expresses only chymase, but not tryptase, and are named MCC (Li et al, J Immunol. 156: 4839-44, 1996). Concerning their functions, besides their role already largely explored as cells involved in immediate hypersensitivity, recent studies have been able to show that mast cells possess two major physiological properties as antigen presenting cells, and as elements highly involved in the anti-infectious defense of the organism (Abraham and Arock, Semin Immunol. 10: 373-381, 1998; Arock and Abraham, Infection Immunity 66: 6030-4, 1998; Galli et al, Curr Opin Immunol. 11: 53-59, 1999).

More recently, the Applicant has discovered that mast cells are involved in numerous pathologies extending much beyond to what one could have previously thought. In this regard, the Applicant filed U.S. Nos. 60/301,408, U.S. 60/601,409, U.S. 60/301,411, U.S. 60/301,407, U.S. 60/301,406, U.S. 60/323,312, U.S. 60/301,410, U.S. 60/323,315, U.S. 60/301,405, U.S. 60/601,409, and U.S. 60/301,404.

It was found that mast cells present in tissues of patients are implicated in or contribute to the genesis of diseases such as autoimmune diseases, allergic diseases, tumor angiogenesis, inflammatory diseases, polyarthritis, inflammatory bowel diseases (IBD), and interstitial cystitis. In all these diseases, it was postulated that mast cells participate in the destruction of tissues by releasing a cocktail of different proteases and mediators such as histamine, proteoglycans, neutral proteases), lipid-derived mediators (prostaglandins, thromboxanes and leucotrienes), and various cytokines (IL-1, IL-2, IL-3, IL-4, IL-5, IL-6, IL-8, TNF-A, GM-CSF, MIP-1a, MIP-1b, MIP-2 and IFN-γ).

For this reason, it has been proposed to target c-kit to deplete the mast cells responsible for these disorders. While this approach is very promising, the Applicant goes even further and propose here to deplete specifically mast cells. Indeed, c-kit is also expressed on others hematopoietic or non hematopoietic cells. Enhancing the specificity for mast cells depletion is of great interest and could open new routes for treating the above indicated diseases. This can be illustrated for example for the treatment of autoimmune diseases. In the art, only immunosuppressive agents have shown a relative efficacy, but they have so much side effects that it jeopardizes the patients global health and it is associated with high rate of morbidity in several cases.

Here, the method defined here-below will allow to provide tailored treatments, compared to what is proposed in the art, since it will target mast cells, a very specific subcomponent of the immune system that is central to the above diseases, but which can be eliminated without affecting the global health of patients.

DESCRIPTION

Therefore, the present invention is aimed at a method for identifying compounds capable of depleting mast cells, wherein said compounds are non-toxic for other hematopoeitic cells that are not mast cells or related cells or cell lines or derived cell lines thereof, such as SCF independent expanded human normal CD34+ cells, comprising the steps consisting of:

a) culturing mast cells in vitro in a suitable culture medium,

b) adding to said culture medium at least one candidate compound to be tested and incubating said cells for a prolonged period of time,

c) measuring the extent to which said compounds promote mast cells death or disrupt, interfere with, or inhibit mast cells growth, and selecting compounds for which mast cells depletion is observed,

d) identifying a subset of compounds selected in step c) that are unable to promote significant death of a cell chosen from other hematopoeitic cells that are not mast cells or related cells or cell lines or derived cell lines thereof, such as SCF independent expanded human normal CD34+ cells.

In other words, the invention relates to a method for identifying compounds capable of depleting mast cells, wherein said compounds are non-toxic for other hematopoeitic cells that are not mast cells or related cells or cell lines or derived cell lines thereof, such as SCF independent expanded human normal CD34+ cells, comprising the step consisting of:

a) providing a culture of mast cells, wherein said mast cells are selected from wild type mast cells and cell lines derived thereof, activated mutant mast cell lines, and activated wild type mast cells and cell lines derived thereof,

b) contacting the culture of said cells with at least one candidate compound under conditions allowing growth and/or survival of mast cells, measuring the level of cell death in the presence of the candidate compound; and comparing the level of cell death in the presence of the candidate compound to the level of cell death in the absence of the candidate compound, wherein an increase in the level of cell death in the presence of the candidate compound is indicative of the mast cells depletion ability of the candidate compound,

c) providing a culture of at least one cell other than mast cells, wherein said cell is selected from hematopoeitic cells that are not mast cells or related cells or cell lines or derived cell lines thereof, such as SCF independent expanded human normal CD34+ cells,

d) contacting the culture of said cells with at least one compound identified in step b) under conditions allowing growth and/or survival of a cell depicted in step c), measuring the level of cell death in the presence of said compound; and comparing the level of cell death in the presence of the compound to the level of cell death in the absence of the compound, wherein no significant increase in the level of cell death in the presence of said compound is indicative of mast cells depletion specificity of said compound versus at least another hematopoeitic cell.

Among the Mast Cells that can be Used in Frame with the Method Depicted Above, We Can Cite:

SCF Dependant Cells:

a) cells originating from blood obtained from human umbilical vein. In this regard, heparinated blood from umbilical vein is centrifuged on a Ficoll gradient so as to isolate mononucleated cells from other blood components. CD34+ precursor cells are then purified from the isolated cells mentioned above using the immunomagnetic selection system MACS (Miltenyi biotech). CD34+cells are then cultured at 37° C. in 5% CO₂ atmosphere at a concentration of 10⁵ cells per ml in the medium MCCM (α-MEM supplemented with L-glutamine, penicillin, streptomycin, 5 10⁻⁵ M β-mercaptoethanol, 20% veal fœtal serum, 1% bovine albumin serum and 100 ng/ml recombinant human SCF. The medium is changed every 5 to 7 days. The percentage of mast cells present in the culture is assessed each week, using May-Grünwal Giemsa or Toluidine blue coloration. Anti-tryptase antibodies can also be used to detect mast cells in culture. After 10 weeks of culture, a pure cellular population of mast cells (>98%) is obtained.

b) Cell lines derived from cells obtained in a). It is possible using standard procedures to prepare vectors expressing c-kit for transfecting the cell lines established as mentioned above. The cDNA of human c-kit has been described in Yarden et al., (1987) EMBO J. 6 (11), 3341-3351. The coding part of c-kit (3000 bp) can be amplified by PCR and cloned, using the following oligonucleotides:

(SEQ ID No2)

-5′AAGAAGAGATGGTACCTCGAGGGGTGACCC3′ sens

(SEQ ID No3)

-5′CTGCTTCGCGGCCGCGTTAACTCTTCTCAACCA3′ antisens

The PCR products, digested with Not1 and Xho1, has been inserted using T4 ligase in the pFlag-CMV vector (SIGMA), which vector is digested with Not1 and Xho1 and dephosphorylated using CIP (Biolabs). The pFlag-CMV-c-kit is used to transform bacterial clone XL1-blue. The transformation of clones is verified using the following primers:

| | |
|---|---|
| -5′AGCTCGTTTAGTGAACCGTC3′ sens, | (SEQ ID No4) |
| -5′GTCAGACAAAATGATGCAAC3′ antisens. | (SEQ ID No5) |

Directed mutagenesis is performed using relevant cassettes is performed with routine and common procedure known in the art.

The vector Migr-1 (ABC) can be used as a basis for constructing retroviral vectors used for transfecting mature mast cells. This vector is advantageous because it contains the sequence coding for GFP at the 3′ and of an IRES. These features allow to select cells infected by the retrovirus using direct analysis with a fluorocytometer. As mentioned above, the N-terminal sequence of c-kit c-DNA can be modified so as to introduce a Flag sequence that will be useful to discriminating heterogeneous from endogenous c-kit.

c) Mast Cell Lines

BaF3 mouse cells expressing wild-type or mutated form of c-kit (in the juxtamembrane and in the catalytic sites) are described in Kitayama et al, (1996), Blood 88, 995-1004 and Tsujimura et al, (1999), Blood 93, 1319-1329. This cell line can be grown in RPMI 1640 medium supplemented with penicillin, streptomycin, L-glutamine, 10% fetal bovine serum (FBS) and IL-3.

IC-2 mouse cells expressing either c-kit^{WT} or c-kit^{D814Y} are presented in Piao et al, (1996), Proc. Natl. Acad. Sci. USA 93, 14665-14669.

d) IL-3 Independent Cell Lines are:

HMC-1, a factor-independent cell line derived from a patient with mast cell leukemia, expresses a juxtamembrane mutant c-kit polypeptide that has constitutive kinase activity (Furitsu T et al, J Clin Invest. 1993;92:1736-1744; Butterfield et al, Establishment of an immature mast cell line from a patient with mast cell leukemia. Leuk Res. 1988;12:345-355 and Nagata et al, Proc Natl Acad Sci USA. 1995;92:10560-10564).

P815 cell line (mastocytoma naturally expressing c-kit mutation at the 814 position) has been described in Tsujimura et al, (1994), Blood 83, 2619-2626. This cell lines is available at ATCC under the accession number TIB-64.

e) Other Mast Cell Lines Available at ATCC

| ATCC N° | Organism | Designation |
|---|---|---|
| CRL-2034 | Mus musculus (mouse) | 10P2 |
| CRL-2036 | Mus musculus (mouse) | 10P12 |
| CRL-2037 | Mus musculus (mouse) | 11P0-1 |

Therefore, in a preferred embodiment, the invention is directed to the above mentioned method, wherein mast cells are chosen from isolated mast cells and cell lines derived thereof, BaF3, IC-2 mouse cells, HMC-1, P815 available at ATCC under the accession number TIB-64, 10P2 available at ATCC under the accession number CRL-2034, 10P12 available at ATCC under the accession number CRL-2036, 11p0-1 available at ATCC under the accession number CRL-2037, and cell lines derived thereof.

In addition, in connection with the method according to the invention, mast cells can be selected from MCC, MCTC, MCT.

In another preferred embodiment, hematopoeitic cells that are not mast cells or related cells or cell lines can be selected from the group consisting of:

human T lymphocyte Jurkat cell line (ATCC N° TIB-152 and cell lines derived thereof),

the human B lymphocyte Daudi or Raji cell line (ATCC N° CCL-213 and CCL-86 respectively and cell lines derived thereof),

the human monocytic U 937 cell line (ATCC N° CRL-1593.2) and,

the human HL-60 cell line (ATCC N° CCL-240) and cell lines derived thereof ATCC N° CRL-2258 and CRL-2392).

As explicated above, the method can be conducted with either one or several of these hematopoeitic cells. Preferred compounds are those who demonstrate the greatest efficacy and specificity for mast cells versus other hematopoeitic cells.

In still another preferred embodiment, control cells are selected from normal human CD34+ cells that are expanded in a culture medium comprising a cocktail of cytokine except SCF. Preferred compounds are those who demonstrate the greatest efficacy and specificity for mast cells versus these SCF independent CD34+ cells.

Concerning efficacy, the compounds of the invention are selected for their ability to deplete mast cells at a concentration below 10 μM, preferably below 5, 4, 2 or 1 μM.

Best compounds are a subset the above indicated compounds, which do not affect significantly the viability of hematopoeitic cells other than mast cells at concentration ranging from 1, 2, 3, 4, 5 μM to 10 μM. Among these compounds, the invention more particularly directed to the ones for which no loss of viability is observed at concentrations ranging from 10 to 15 μM, 15 to 20 μM or 20 to 40 μM.

Ratios Efficacy/Selectivity can be addressed with the above data using the formula:

Ratios E/S=IC50 mast cells/IC50 hematopoeitic cells other than mast cells.

Best compounds are those exhibiting the lowest E/S ratios, for example E/S ratios ranging from 1/1000 to 1/5, 1/1000 to 1/100, 1/100 to 1/50, 1/100 to 1/10, 1/50 to 1/10, 1/25 to 1/10, or 1/20 to 1/5.

The cell death assay can further comprise a cell proliferation assay, a cell viability assay and/or an apoptosis assay.

For example, the extent of cell death can be measured by 3H thymidine incorporation, the trypan blue exclusion method, using propidium iodide or by the ⁵“Cr-release assay.

Alternatively, the extent of cell death can be determined by a test of intracellular esterase activity, and a test of plasma membrane integrity, preferably using fluorescent calcein and ethidium homodimer-1. These tests are described in J. Neurosci 15, 5389 (1995), in J. Cell Sci. 106, 685 (1993). Detailed protocols are given in the Molecular Probes Catalogue product number L-3224 (Live/Dead® Kits) incorporated herein by reference. Basically, calcein AM is the cell-permeant esterase substrate, which is nonfluorescent until converted by enzymatic activity to highly fluorescent calcein. It remains within living cells exhibiting an intense green fluorescence. Ethidium homodimer-1 fluorescence is enhanced upon binding nucleic acids. A bright red fluorescence is emitted. This dye cannot cross intact plasma membranes but it enters into dead cells. Thus, living cells are green, while dead cells emits a red fluorescence. This technique coupled with CDD camera and plate readers leads to high through put screening.

In another embodiment, the extent of cell death is determined by discriminating between living and dead cells using DiOC₁₈ and propidium iodide. Protocols are described in details in the Molecular Probes Catalogue product number L-7010 (Live/Dead® Kits) incorporated herein by reference.

In still another embodiment, cell death can be determined using the Caspase activity test. Caspase is a key player in the activation of apoptosis. The Molecular probe kit E-13183 (EnzCheck Caspase-3 Assay kit®, Molecular Probe) is particularly useful for testing Jurkat cells. Phosphatidyl exposure can also be used in this regard. This method has been employed in Dan S, et al, Selective induction of apoptosis in Philadelphia chromosome-positive chronic myelogenous leukemia cells by an inhibitor of BCR-ABL tyrosine kinase CGP 57148. Cell Death Differ. 1998;5:710-715.

In still another embodiment, cell death can be determined using the Mitochondrial membrane depolarization test using the JC-1 or JC-9 cationic dyes of Molecular Probe, which have been described as a useful indicator in HL-60 cells.

For cell proliferation assays, it can be performed using MTS tetrazolium (Cell Titer96 Aqueous; Promega, Madison, Wis.). This test allows to measure the numbers of viable cells.

In all the above mentioned cell death tests, the invention encompasses fluorometric assays of cell viability and cytotoxicity using a fluorescence microscope, a fluorometer, a fluorescence microplate reader and/or a flow cytometer.

Furthermore, the Applicant has shown in the previously filed U.S. No. 60/301,404 that c-kit is a target of interest for depleting mast cells. It is now more specifically proposed to test inhibitors of the downstream signaling pathways of this receptor. Indeed, among all the tyrosine kinases involved in transducing the signals, one or several of them may be more specific to or upregulated in mast cells versus other hematopoietic cells that are not mast cells.

In this regard, compounds to be tested can be selected from inhibitors of tyrosine kinases, such as Akt, c-Cbl, CRKL, Doc, p125 Fak, Fyn, Grap, Jak2, Lyn, MAPK, MATK, P13-K, PLC-γ, Raf1, Ras, SHP-1, SHP2 (Syp), Tec, Vav and Flt-3 (see Table 1 below).

TABLE 1
Molecules interacting with the intracellular portion of the human c-kit and/
or activated in response to SCF.

| Molecules | Human cells |
|---|---|
| Akt | 293, U2OS, BHX21, HeLa |
| c-Cb1 | MO7e, TF-1 |
| CRKL | MO7e |
| Doc | MO7e |
| p125 Fak | TF-1 |
| Fyn | MO7e |
| Grap | MO7e, TF-1, K562 |
| Jak2 | MO7e, TF-1 |
| Lyn | MO7e, Normal progenitors |
| MAPK | melano 501 mel |
| MATK (CHK) | CMK |
| PI3-K | 293, U2OS, BHX21, HeLa |
| PLC-γ | MO7e |
| Raf1 | MO7e |
| Ras | MO7e |
| SHP-1 | MO7e |
| SHP2 (Syp) | MO7e |
| Tec | MO7e |
| Vav | MO7e, TF-1 |

Compounds of interest include but are not limited to indolinone, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

The invention also relates to a compound obtainable by the method depicted above, wherein said compound is capable of depleting mast cells and has no significant toxicity for other hematopoietic cells. Preferably, such compounds has an E/S ratio ranging from ranging from 1/1000 to 1/5, 1/1000 to 1/100, 1/100 to 1/50, 1/100 to 1/10, 1/50 to 1/10, 1/25 to 1/10, or 1/20 to 1/5.

Another aspect of the invention is directed to the use of said compound to manufacture a medicament. Such medicament can take the form of a pharmaceutical compositions for oral administration, which can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Such medicament can take the form of a pharmaceutical or cosmetic compositions for topical administration. Such compositions according to the invention may be presented in the form of a gel, paste, ointment, cream, lotion, liquid suspension aqueous, aqueous-alcoholic or, oily solutions, or dispersions of the lotion or serum type, or anhydrous or lipophilic gels, or emulsions of liquid or semi-solid consistency of the milk type, obtained by dispersing a fatty phase in an aqueous phase or vice versa, or of suspensions or emulsions of soft, semi-solid consistency of the cream or gel type, or alternatively of microemulsions, of microcapsules, of microparticles or of vesicular dispersions to the ionic and/or nonionic type. These compositions are prepared according to standard methods.

The composition according to the invention comprises any ingredient commonly used in dermatology and cosmetic. It may comprise at least one ingredient selected from hydrophilic or lipophilic gelling agents, hydrophilic or lipophilic active agents, preservatives, emollients, viscosity enhancing polymers, humectants, surfactants, preservatives, antioxidants, solvents, and fillers, antioxidants, solvents, perfumes, fillers, screening agents, bactericides, odor absorbers and coloring matter.

As oils which can be used in the invention, mineral oils (liquid paraffin), vegetable oils (liquid fraction of shea butter, sunflower oil), animal oils, synthetic oils, silicone oils (cyclomethicone) and fluorinated oils may be mentioned. Fatty alcohols, fatty acids (stearic acid) and waxes (paraffin, carnauba, beeswax) may also be used as fatty substances.

As emulsifiers which can be used in the invention, glycerol stearate, polysorbate 60 and the PEG-6/PEG-32/glycol stearate mixture are contemplated. As hydrophilic gelling agents, carboxyvinyl polymers (carbomer), acrylic copolymers such as acrylate/alkylacrylate copolymers, polyacrylamides, polysaccharides such as hydroxypropylcellulose, clays and natural gums may be mentioned, and as lipophilic gelling agents, modified clays such as bentones, metal salts of fatty acids such as aluminum stearates and hydrophobic silica, or alternatively ethylcellulose and polyethylene may be mentioned.

As hydrophilic active agents, proteins or protein hydrolysates, amino acids, polyols, urea, allantoin, sugars and sugar derivatives, vitamins, starch and plant extracts, in particular those of Aloe vera may be used.

As lipophilic active, agents, retinol (vitamin A) and its derivatives, tocopherol (vitamin E) and its derivatives, essential fatty acids, ceramides and essential oils may be used. These agents add extra moisturizing or skin softening features when utilized.

In addition, a surfactant can be included in the composition so as to provide deeper penetration of the compound capable of depleting mast cells, such as a tyrosine kinase inhibitor, preferably a c-kit inhibitor.

Among the contemplated ingredients, the invention embraces penetration enhancing agents selected for example from the group consisting of mineral oil, water, ethanol, triacetin, glycerin and propylene glycol; cohesion agents selected for example from the group consisting of polyisobutylene, polyvinyl acetate and polyvinyl alcohol, and thickening agents.

Chemical methods of enhancing topical absorption of drugs are well known in the art. For example, compounds with penetration enhancing properties include sodium lauryl sulfate (Dugard, P. H. and Sheuplein, R. J., “Effects of Ionic Surfactants on the Permeability of Human Epidermis: An Electrometric Study,” J. Ivest. Dermatol., V.60, pp. 263-69, 1973), lauryl amine oxide (Johnson et. al., U.S. Pat. No. 4,411,893), azone (Rajadhyaksha, U.S. Pat. Nos. 4,405,616 and 3,989,816) and decylmethyl sulfoxide (Sekura, D. L. and Scala, J., “The Percutaneous Absorption of Alkylmethyl Sulfides,” Pharmacology of the Skin, Advances In Biology of Skin, (Appleton-Century Craft) V. 12, pp. 257-69, 1972). It has been observed that increasing the polarity of the head group in amphoteric molecules increases their penetration-enhancing properties but at the expense of increasing their skin irritating properties (Cooper, E. R. and Bemer, B., “Interaction of Surfactants with Epidermal Tissues: Physiochemical Aspects,” Surfactant Science Series, V. 16, Reiger, M. M. ed. (Marcel Dekker, Inc.) pp. 195-210, 1987).

A second class of chemical enhancers are generally referred to as co-solvents. These materials are absorbed topically relatively easily, and, by a variety of mechanisms, achieve permeation enhancement for some drugs. Ethanol (Gale et. al., U.S. Pat. No. 4,615,699 and Campbell et. al., U.S. Pat. Nos. 4,460,372 and 4,379,454), dimethyl sulfoxide (U.S. Pat. Nos. 3,740,420 and 3,743,727, and U.S. Pat. No. 4,575,515), and glycerine derivatives (U.S. Pat. No. 4,322,433) are a few examples of compounds which have shown an ability to enhance the absorption of various compounds.

The pharmaceutical compositions of the invention can also be intended for administration with aerosolized formulation to target areas of a patient's respiratory tract.

Devices and methodologies for delivering aerosolized bursts of a formulation of a drug is disclosed in U.S. Pat. No. 5,906,202. Formulations are preferably solutions, e.g. aqueous solutions, ethanoic solutions, aqueous/ethanoic solutions, saline solutions, colloidal suspensions and microcrystalline suspensions. For example aerosolized particles comprise the active ingredient mentioned above and a carrier, (e.g., a pharmaceutically active respiratory drug and carrier) which are formed upon forcing the formulation through a nozzle which nozzle is preferably in the form of a flexible porous membrane. The particles have a size which is sufficiently small such that when the particles are formed they remain suspended in the air for a sufficient amount of time such that the patient can inhale the particles into the patient's lungs.

The invention encompasses systems described in U.S. Pat. No. 5,556,611:

liquid gas systems (a liquefied gas is used as propellent gas (e.g. low-boiling FCHC or propane, butane) in a pressure container,

suspension aerosol (the active substance particles are suspended in solid form in the liquid propellent phase),

pressurized gas system (a compressed gas such as nitrogen, carbon dioxide, dinitrogen monoxide, air is used.

Thus, according to the invention the pharmaceutical preparation is made in that the active substance is dissolved or dispersed in a suitable nontoxic medium and said solution or dispersion atomized to an aerosol, i.e. distributed extremely finely in a carrier gas. This is technically possible for example in the form of aerosol propellent gas packs, pump aerosols or other devices known per se for liquid misting and solid atomizing which in particular permit an exact individual dosage. Therefore, the invention is also directed to aerosol devices comprising the compound as defined above and such a formulation, preferably with metered dose valves.

The pharmaceutical compositions of the invention can also be intended for intranasal administration. In this regard, pharmaceutically acceptable carriers for administering the compound to the nasal mucosal surfaces will be readily appreciated by the ordinary artisan. Such carriers are disclosed, simply by way of example, by Remington's Pharmaceutical Sciences” 16th edition, 1980, Ed. By Arthur Osol, the disclosure of which is incorporated herein by reference.

The selection of appropriate carriers depends upon the particular type of administration that is contemplated. For administration via the upper respiratory tract. The composition can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or upper respiratory administration.

Common intranasal carriers include nasal gels, creams, pastes or ointments with a viscosity of, e.g., from about 10 to about 3000 cps, or from about 2500 to 6500 cps, or greater, may also be used to provide a more sustained contact with the nasal mucosal surfaces. Such carrier viscous formulations may be based upon, simply by way of example, alkylcelluloses and/or other biocompatible carriers of high viscosity well known to the art (see e.g., Remington's, cited supra. A preferred alkylcellulose is, e.g., methylcellulose in a concentration ranging from about 5 to about 1000 or more mg per 100 ml of carrier. A more preferred concentration of methyl cellulose is, simply by way of example, from about 25 to about mg per 100 ml of carrier.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. For nasal administration of solutions or suspensions according to the invention, various devices are available in the art for the generation of drops, droplets and sprays.

A premeasured unit dosage dispenser including a dropper or spray device containing a solution or suspension for delivery as drops or as a spray is prepared containing one or more doses of the drug to be administered and is another object of the invention. The invention also includes a kit containing one or more unit dehydrated doses of the compound, together with any required salts and/or buffer agents, preservatives, colorants and the like, ready for preparation of a solution or suspension by the addition of a suitable amount of water.

In still another aspect, the invention is aimed at a method for treating a disease selected from autoimmune diseases, allergic diseases, bone loss, tumor angiogenesis, inflammatory diseases, inflammatory bowel diseases (IBD), interstitial cystitis, mastocytosis, infections diseases, and CNS disorders comprising administering a compound obtainable from a method depicted above to a mammal in need of such treatment.

In a further aspect, the invention contemplates a method for promoting hair growth and hair color revival comprising administering a compound obtainable from a method from a method depicted above to a human need of such treatment.

In a still further aspect, the invention embraces a method as defined above for treating multiple sclerosis, psoriasis, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, Sjogren's syndrome, nodular panarteritis, autoimmune enteropathy, proliferative glomerulonephritis, active chronic hepatitis and chronic fatigue syndrome.

In a still further aspect, the invention embraces a method as defined above for treating graft-versus-host disease or graft rejection in any organ transplantation including kidney, pancreas, liver, heart and lung.

In another aspect, the invention embraces a method as defined above for treating subepidermal blistering disorders such as aphthous ulcers, and several bullous diseases such as pemphigus, bullous pemphigoid and cicatricial pemphigoid. This method can further comprises administering at least one antibiotic, preferably selected from dapsone, azathioprine, erythromycin, propionylerythromycin, neomycin, gentomycin, tobramycin, and mechlocycline.

The invention also relates to a method as described above for treating asthma, allergic rhinitis, allergic sinusitis, anaphylactic syndrome, urticaria, angioedema, atopic dermatitis, allergic contact dermatitis, erythema nodosum, erythema multiforme, cutaneous necrotizing venulitis, insect bite skin inflammation and blood sucking parasitic infestation.

The invention also relates to a method as described above for treating skin allergic disorders such as urticaria, atopic dermatitis, allergic contact dermatitis, erythema nodosum, erythema multiforme, cutaneous necrotizing venulitis, insect bite skin inflammation and blood sucking parasitic infestation especially in dogs and cats.

Here, the compound can be administered with aerosolized formulations to target areas of a patient's respiratory tract, or with intranasal or topical formulations accordingly.

In yet another aspect, the invention embraces a method as defined above for treating tumor angiogenesis in human.

The invention also concerns the method as depicted above for treating skin disorders in human associated with mastocytosis, notably cutaneous mastocytosis including urticaria pigmentosa, diffuse cutaneous mastocytosis, solitary mastocytoma and bullous, erythrodermic and teleangiectatic mastocytosis, as well as for treating category IV mastocytosis including mast cell leukemia.

A particular embodiment is directed to the treatment of dog mastocytoma.

In yet another aspect, the invention embraces the method as depicted above for treating treating inflammatory bowel diseases (IBD), such as Crohn's disease, mucositis, ulcerative colitis, and necrotizing enterocolitis.

The invention also contemplates the method as depicted above for treating interstitial cystitis in human, for treating bacterial infections in mammalian, especially in human, preferably for the treatment of recurrent bacterial infections, resurging infections after asymptomatic periods such as bacterial cystitis.

More particularly, the invention can be practiced for treating FimH expressing bacteria infections such as Gram-negative enterobacteria including E. coli, Klebsiella pneumoniae, Serratia marcescens, Citrobactor freudii and Salmonella typhimurium.

In this method for treating bacterial infection, further administration of at least one antibiotic selected bacitracin, the cephalosporins, the penicillins, the aminoglycosides, the tetracyclines, the streptomycins and the macrolide antibiotics such as erythromycin; the fluoroquinolones, actinomycin, the sulfonamides and trimethoprim, is of interest.

The invention also contemplates the method as depicted above for treating bone loss such as osteoporosis, including post menopausal osteoporosis, senile osteoporosis, and glucocorticoid-induced osteoporosis, osteitis fibrosa cystica, renal osteodystrophy, osteosclerosis, osteopenia, osteomalacia, fibrogenesis-imperfecta ossium, and Paget's Disease.

In a particular embodiment, the invention relates to the method as depicted above for treating inflammatory disorders such as rheumatoid arthritis, conjunctivitis, rheumatoid spondylitis, osteoarthritis, gouty arthritis, polyarthritis, and other arthritic conditions as well as pain associated with these inflammatory diseases.

Utility of the invention will be further illustrated with the hereinafter detailed description. Indeed, differences in signal transduction with c-kit wild or mutated c-kit has been observed. This could lead to target specific second messagers that are specifically present, activated or upregulated in mast cells or pathways that are repressed, not present or inactivated in hematopoietic cells other than mast cells.

Signal Transduction Induced by Activation of Normal C-Kit.

SCF is an essential growth factor in hematopoiesis since it synergizes with almost all the hematopoietic growth factors, except M-CSF, to induce in vitro hematopoiesis, Metcalf, D. (1993) The cellular basis for enhancement interactions between stem cell factor and the colony stimulating factors. Stem Cells (Dayt) 11 Suppl 2, 1-11. This factor is produced by bone marrow stromal cells, and acts through interaction with its receptor, c-kit, Ratajczak, M. Z et al, (1992) Role of the KIT protooncogene in normal and malignant human hematopoiesis. Proc. Natl. Acad. Sci. USA 89, 1710-1714. As previously noticed, the c-kit receptor is a glycoprotein of 145 kDa and belongs to the type III tyrosine kinase subfamily, characterized by the presence of five Ig-like domains in the extracellular part of the molecule and by an interkinase sequence that splits the intracytoplasmic domain into the adenosine triphosphate (ATP)-binding domain and the phosphotransferase domain. C-kit is strongly expressed by CFU-GEMM, BFU-E and by progenitors and mature cells of the mast cell lineage, Katayama N. et al, (1993) Stage-specific expression of c-kit protein by murine hematopoietic progenitors. Blood 82, 2353-2360.

Ligand binding to c-kit results in activation of the catalytic function, resulting in autophosphorylation of tyrosine residues of the cytoplasmic domain. These phosphotyrosine residues become docking sites for various cytoplasmic signaling molecules containing SH2 domain. C-kit activates canonical signal transduction pathways common to many growth factor receptors, including those depending on PI3-kinase, ras and JAK2. Molecules known to associate with c-kit in vivo or in vitro include p85 subunit of P13-kinase, multiple Src family members, Lyn and Fyn, Vav, Grb2, SHP-1, SHP-2, PKC, MATK (CHK) and Socs1, while there are divergent data concerning PLC-γ, GTPase activating Protein of ras (GAP) and JAK2. Additional molecules are activated or phosphorylated in response to c-kit activation: Shc, Tec, Vav DP/GTP exchange factor, raf-1, MAPK, Akt, CRKL, p120 Cbl, and Doc. Recent studies performed in various cell systems have yielded divergent results regarding the substrates that associate with and are phosphorylated by c-kit. These discrepancies might reflect either differences in experimental methods or functionally relevant variations in substrate expression profiles of individual cell types, which could be the basis of distinct signals and cell type specific responses mediated by the same ligand/receptor system. For these reasons, we choose to describe the data obtained regarding c-kit signaling in various cellular contexts.

The first initiator of signalization is the ligand induced-dimerization of c-kit, which induces intrinsic tyrosine kinase activity of c-kit, resulting in transphosphorylation at critical tyrosine residues. Moreover, in response to ligand stimulation, c-kit appears to be phosphorylated on serine residues by PKC, which inhibits c-kit autophosphorylation, Katayama, N et al, (1993) Stage-specific expression of c-kit protein by murine hematopoietic progenitors. Blood 82, 2353-2360.

One of the most efficient associations with c-kit, observed in various cell types, is contracted by SH2 domain of p85 subunit of P13-kinase, Lev, S et al. (1992) Interkinase domain of kit contains the binding site for phosphatidylinositol 3′ kinase. Proc. Natl. Acad. Sci. USA 89, 678-682 and Rottapel, R. et al (1991) The Steel/W transduction pathway: kit autophosphorylation and its association with a unique subset of cytoplasmic signaling proteins is induced by the Steel factor. Mol. Cell. Biol. 11, 3043-3051.via the phosphorylated tyrosine residue 719 of murine c-kit or tyrosine 721 of human c-kit, Serve, H et al, (1994) Tyrosine residue 719 of the c-kit receptor is essential for binding of the P85 subunit of phosphatidylinositol (PI) 3-kinase and for c-kit-associated PI3-kinase activity in COS-1 cells. J. Biol. Chem. 269, 6026-6030. C-kit signalisation has been studied in human hematopoietic cells, mainly in MO7e and CMK, two megakaryocytic cell lines (Table 1 above).

In these cells, SCF induces activation and/or recruitment of major kinases such as P13-kinase, Src kinases (Fyn and Lyn) and JAK2, and various adaptators molecules, Grb2, Grap, Vav, CRKL via their SH2 domain. These events result in formation of various molecular associations via SH2, SH3 or PH domains, which in turn start activation of different pathways. Ras pathway was showed to be activated in response to SCF stimulation, leading to interaction between Ras and Raf-1, thus initiating MAPKinase cascade, Tauchi, T et al, (1994) The ubiquitously expressed Syp phosphatase interacts with c-kit and Grb2 in hematopoietic cells. J. Biol. Chem. 269, 25206-25211.

Indeed, Grap, an adaptator molecule, interacts with ligand-activated c-kit through its SH2 domain and is associated with a ras guanine nucleotide exchange factor, mSos1, through its SH3 domain, coupling signals from receptor and cytoplasmic tyrosine kinase to the ras signaling pathway, Feng, G. S et al, (1996) Grap is a novel SH3-SH2-SH3 adaptor protein that couples tyrosine kinases to the Ras pathway. J. Biol. Chem. 271, 12129-12132. Another adaptator molecule related to Grap, Grb2, interacting via its SH2 domain with a phosphorylated tyrosine residue of c-kit, may recruit c-Cb1 and Shc, Brizzi, M. F et al, (1996) Discrete protein interactions with the Grb2/c-Cbl complex in SCF- and TPO-mediated myeloid cell proliferation. Oncogene 13, 2067-2076 and Wisniewski, D., Strife, A., Clarkson, B. (1996) c-kit ligand stimulates tyrosine phosphorylation of the c-Cbl protein in human hematopoietic cells. Leukemia 10, 1436-1442. After activation, kinase may either play the role of adaptator molecule such as PI3-kinase interacting with c-Cbl and CRKL, Sattler, M. et al., (1997) Steel factor induces tyrosine phosphorylation of CRKL and binding of CRKL to a complex containing c-kit, phosphatidylinositol 3-kinase, and p120(CBL). J. Biol. Chem. 272, 10248-10253 or the role of kinase such as PI3-kinase phosphorylating Akt, Blume-Jensen, P et al, (1998) The kit receptor promotes cell survival via activation of PI 3-kinase and subsequent Akt-mediated phosphorylation of Bad on Ser136. Curr. Biol. 8, 779-782.

In few cases, interaction and/or activation is described without connection with any known signaling pathway. This is the case for Tec, Tang, B et al, (1994) Tec kinase associates with c-kit and is tyrosine phosphorylated and activated following stem cell factor binding. Mol. Cell. Biol. 14, 8432-8437, for MATK, Jhun, B. H. et al, (1995) The MATK tyrosine kinase interacts in a specific and SH2-dependent manner with c-Kit. J. Biol. Chem. 270, 9661-9666, Vav, Alai, M et al, (1992) Steel factor stimulates the tyrosine phosphorylation of the proto-oncogene product, p95vav, in human hemopoietic cells. J. Biol. Chem. 267, 18021-18025, and Doc, Carpino, N. et al, (1997) p62(dok): a constitutively tyrosine-phosphorylated, GAP-associated protein in chronic myelogenous leukemia progenitor cells. Cell 88, 197-204.

Unexpectedly, JAK-STAT pathway is poorly described during c-kit activation. JAK2, a cytosolic tyrosine kinase essential for non tyrosine kinase cytokine receptor superfamily signaling, has been described physically associated with c-kit, prior to ligand activation, and phosphorylated on tyrosine residues in response to SCF, Brizzi, M. et al, (1994) Convergence of signaling by interleukin-3, granulocyte-macrophage colony-stimulating factor, and mast cell growth factor on JAK2 tyrosine kinase. J. Biol. Chem. 269, 31680-31684, Weiler, S. R et al, (1996) JAK2 is associated with the c-kit proto-oncogene product and is phosphorylated in response to stem cell factor. Blood 87, 3688-3693 and Linnekin, D. et al, (1996) JAK2 is constitutively associated with c-Kit and is phosphorylated in response to stem cell factor. Acta Haematol. 95, 224-228., or not associated with c-kit. In addition, SCF activates cytosolic transcription factors like STAT1 in MO7e cell line, Deberry, C. et al, (1997) Statl associates with c-kit and is activated in response to stem cell factor. Biochem. J. 327 (Pt 1), 73-80. C-kit signalisation has been also examined in various non hematopoietic human cell lines. Blume-Jensen et al. demonstrated that SCF induced activation of Akt and mediated phosphorylation of Serine residue 136 of Bad in a P13-kinase-dependent manner, Blume-Jensen, P. et al, (1998). In vitro experiments performed on embryonic fibroblasts indicate that P13-kinase and PLC-γ compete for association with tyrosine residue 721 of human c-kit, with p85/PI3-kinase exhibiting higher affinity, Herbst, R et al, (1995) Formation of signal transfer complexes between stem cell and platelet-derived growth factor receptors and SH2 domain proteins in vitro. Biochemistry 34, 5971-5979. In H526 cell line (Small cell lung carcinoma, SCLC), SCF induced activation of Src-kinase, Lck, and its interaction with the juxtamembrane domain of c-kit, Krystal, G. W et al, (1998) Lck associates with and is activated by Kit in a small cell lung cancer cell line: inhibition of SCF-mediated growth by the Src family kinase inhibitor PP1. Cancer Res. 58, 4660-4666. In 501 mel, a human melanoma cell line, Hemesath et al. (1998), Nature 391, 298-301 described that SCF stimulation resulted in activation of MAPK which, in turn, phosphorylated transcription factor microphtalmia (Mi), upregulating Mi transactivation via interaction with p300/CBP.

Interestingly, interconnection between c-kit and integrin signaling pathways was observed in TF-1 cell line. SCF induces spreading of fibronectin-adherent TF-1 cells and enhances tyrosine phosphorylation of pp125 FAK in a dose-dependent manner, when compared to the level of tyrosine phosphorylation of pp125 FAK in the absence of SCF. These effects depend on a worthmannin-, integrin activation-sensitive pathways.

Regarding c-kit deactivation, two main pathways have been described in different cellular contexts. In hematopoietic cells, one pathway involves SHP-1, a tyrosine phosphatase, interacting with c-kit probably at 569 tyrosine phosphorylated residue, which down-regulates tyrosine residue phosphorylation state of c-kit, Yi, T., Ihle, J. N. (1993) Association of hematopoietic cell phosphatase with c-Kit after stimulation with c-Kit ligand. Mol. Cell. Biol. 13, 3350-3358, Lorenz, U. et al, (1996) Genetic analysis reveals cell type-specific regulation of receptor tyrosine kinase c-Kit by the protein tyrosine phosphatase SHP1. J. Exp. Med. 184, 1111-1126, Paulson, R. et al, (1996) Signalling by the W/Kit receptor tyrosine kinase is negatively regulated in vivo by the protein tyrosine phosphatase Shp1. Nat. Genet. 13, 309-315, and Kozlowski, M et al, (1998) SHP-1 binds and negatively modulates the c-Kit receptor by interaction with tyrosine 569 in the c-Kit juxtamembrane domain. Mol. Cell. Biol. 18, 2089-2099.

The role of phosphatase SHP-2 (Syp) is less clear. It has been shown that SHP-2 associated with activated c-kit in MO7e cell line via its SH2 domain, became phosphorylated and complexed with Grb2, Tauchi T. et al, (1994). This connection to Grb2 could lead to ras/MAPkinase pathway activation and to cell proliferation. By contrast, it has been shown, in BA/F3 cells expressing c-kit, that SHP-2 association to c-kit Y567F is markedly reduced. In this case, an hyperproliferative response to SCF was observed, suggesting that SHP-2 downregulates SCF-induced proliferation, Kozlowski, M. et al, (1998).

Activation and deactivation of human c-kit have been also studied in porcine endothelial cells (PAE). Activation of P13-kinase, PLC-γ and Raf/MAPKinase cascade was described in response to SCF in PAE cells transfected with human c-kit. In these cells, a first negative feedback loop is the PI3-K, PLD and PKC pathway which leads to phosphorylation at 741 and 746 serine residues of c-kit. A second deactivation pathway is P13-kinase-induced PLD activation and phosphatidylcholine (PtdCho)-specific phospholipase D activation, (PtdCho)-PLD, that generated phosphatidic acid (PtdH), metabolized into diacylglycerol (DAG), an activator of PKC and a precursor of arachidonic acid (D4Ach), Kozawa, 0 et al, (1997) Involvement of phosphatidylinositol 3′-kinase in stem-cell-factor-induced phospholipase D activation and arachidonic acid release. Eur. J. Biochem. 248, 149-155. These authors also showed that SCF induced PLA2 activation, a second pathway generating D4Ach.

In murine bone marrow-derived mast cells (BMMC), it has been demonstrated that SCF induces i) P13-kinase activation, which in turn stimulates Rac-1 and Jnk pathway and ii) binding and phosphorylation of Src kinases Fyn on tyrosine 567, Timokhina, I et al, (1998) Kit signaling through PI 3-kinase and Src kinase pathways: an essential role for Racl and JNK activation in mast cell proliferation. EMBO J. 17, 6250-6262 and Lyn, Suzuki, T. et al, (1998) Essential roles of Lyn in fibronectin-mediated filamentous actin assembly and cell motility in mast cells. J. Immunol. 161, 3694-3701. In rat mast cells isolated from the peritoneal cavity, Koike et al. have shown that SCF induced PLD activation and subsequent release of D4Ach through the protein tyrosine kinase pathway and without activation of the phosphoinositide-specific PLC-γ, Koike, T. et al, (1993) SCF/c-kit receptor-mediated arachidonic acid liberation in rat mast cells. Involvement of PLD activation associated tyrosine phosphorylation. Biochem. Biophys. Res. Commun. 197, 1570-1577 and Koike, T. et al, (1993) Stem cell factor-induced signal transduction in rat mast cells. Activation of phospholipase D but not phosphoinositide-specific phospholipase C in c-kit receptor stimulation. J. Immunol. 151, 359-366. In these cells, Nagai et al. have shown the involvement of P13-kinase, protein tyrosine kinase (PTK) and myosin light chain kinase in SCF induced histamine release, Nagai, S. et al, (1995) Pharmacological study of stem-cell-factor-induced mast cell histamine release with kinase inhibitors. Biochem. Biophys. Res. Commun. 208, 576-581.

Regarding c-kit deactivation in murine cells, another way to decrease SCF signal is the down-modulation of c-kit expression. Yee et al. and Miyazawa et al. have shown that c-kit internalization and ubiquitination is dependent on intact kinase activity of c-kit, Yee, N. et al, (1994) Mechanism of down-regulation of c-kit receptor. Roles of receptor tyrosine kinase, phosphatidylinositol 3′-kinase, and protein kinase C. J. Biol. Chem. 269, 31991-31998 and Miyazawa, K. et al, (1994) Ligand-dependent polyubiquitination of c-kit gene product: a possible mechanism of receptor down modulation in M07e cells. Blood 83, 137-145.

In BMMC, c-kit activates PLC-γ resulting in the hydrolysis of PI4,5 diP into DAG and inositol-1,4,5 trip inducing mobilization of intracellular Ca⁺⁺. This calcium influx seems to be critical for c-kit internalization. Moreover, in the absence of PI3-kinase activation, the c-kit receptor internalizes but remains localized near the inner side of the plasma membrane. Of note, c-kit internalization is completely prevented when both PI3-kinase and Ca⁺⁺ influx are inhibited, Gommerman, J. L. et al, (1997) Phosphatidylinositol 3-kinase and Ca2+ influx dependence for ligand-stimulated internalization of the c-Kit receptor. J. Biol. Chem. 272, 30519-30525.

A novel mediator of downregulation of c-kit-dependent mitogenesis could be Socs-1 (Suppressor of cytokine signaling), De Sepulveda, P. et al, (1999) Socs1 binds to multiple signalling proteins and suppresses Steel factor-dependent proliferation. EMBO J. 18, 904-915. SCF induces synthesis of Socs-1, that binds to c-kit via its SH2 domain.

The mechanism of Socs-1 activity seems to involve its interaction with Grb2 and the negative regulatory N-terminus of Vav, Blechman, J. et al, (1993) Structure-function analyses of the kit receptor for the steel factor. Stem Cells (Dayt) 11 Suppl 2, 12-21.

Molecular Dysfunctions Related to C-Kit Mutations

Enzymatic Functions and C-Kit Mutations

SHP-1 expression is different in IC2/c-kit^{WT} and IC2/c^{D814Y} cells, and this is also the case for other proteins like MMCP-4 and MMCP-6, that are proteases present in the granules of murine mast cells and differentially expressed at various stages of mast cell maturation. Indeed, MMCP-6 transcripts are expressed at low level in IC2/c-kit^{WT} cells in the presence of exogenous SCF, and this level increases as the result of c-kitD^{814Y} expression. MMCP-4 transcripts are not detectable by RT-PCR in IC2/c-kit^{WT} cells, but are abundantly expressed in IC2c-kit^{D814Y} cells. The differences observed between the wild form and the mutant suggest that the signals transduced by c-kit^{WT} stimulated by SCF and by c-kit^{D814Y} are not equivalent: the mutation c-kit^{D814Y} alters not only the proliferation of mast cells but also their stage of maturation.

1. A method for identifying compounds capable of depleting mast cells, wherein said compounds are non-toxic for other hematopoeitic cells that are not mast cells or related cells or cell lines or derived cell lines thereof, such as SCF independent expanded human normal CD34+ cells, comprising the steps consisting of:
a) culturing mast cells in vitro in a suitable culture medium,
b) adding to said culture medium at least one candidate compound to be tested and incubating said cells for a prolonged period of time,
c) measuring the extent to which said compounds promote mast cells death or disrupt, interfere with, or inhibit mast cells growth, and selecting compounds for which mast cells depletion is observed,
d) identifying a subset of compounds selected in step c) that are unable to promote significant death of a cell chosen from other hematopoeitic cells that are not mast cells or related cells or cell lines or derived cell lines thereof, such as SCF independent expanded human normal CD34+ cells.

2. A method for identifying compounds capable of depleting mast cells, wherein said compounds are non-toxic for other hematopoeitic cells that are not mast cells or related cells or cell lines or derived cell lines thereof, such as SCF independent expanded human normal CD34+ cells, comprising the step consisting of:
a) providing a culture of mast cells, wherein said mast cells are selected from wild type mast cells and cell lines derived thereof, activated mutant mast cell lines, and activated wild type mast cells and cell lines derived thereof,
b) contacting the culture of said cells with at least one candidate compound under conditions allowing growth and/or survival of mast cells, measuring the level of cell death in the presence of the candidate compound; and comparing the level of cell death in the presence of the candidate compound to the level of cell death in the absence of the candidate compound, wherein an increase in the level of cell death in the presence of the candidate compound is indicative of the mast cells depletion ability of the candidate compound,
c) providing a culture of at least one cell other than mast cells, wherein said cell is selected from hematopoeitic cells that are not mast cells or related cells or cell lines or derived cell lines thereof, such as SCF independent expanded human normal CD34+ cells,
d) contacting the culture of said cells with at least one compound identified in step b) under conditions allowing growth and/or survival of the cell depicted in step c), measuring the level of cell death in the presence of said compound; and comparing the level of cell death in the presence of the compound to the level of cell death in the absence of the compound, wherein no significant increase in the level of cell death in the presence of said compound is indicative of mast cells depletion specificity of said compound versus at least another hematopoeitic cell.

3. A method according to claim 1 or 2, wherein mast cells are chosen from isolated mast cells and cell lines derived thereof, BaF3, IC-2 mouse cells, HMC-1, P815 available at ATCC under the accession number TIB-64, 10P2 available at ATCC under the accession number CRL-2034, 10P12 available at ATCC under the accession number CRL-2036, 11P0-1 available at ATCC under the accession number CRL-2037, and cell lines derived thereof.

4. A method according to one of claims 1 to 3, wherein other hematopoeitic cells that are not mast cells or related cells or cell lines are selected from the group consisting of human T lymphocyte Jurkat cell line (ATCC N° TIB-152 and cell lines derived thereof), the human B lymphocyte Daudi or Raji cell line (ATCC N° CCL-213 and CCL-86 respectively and cell lines derived thereof), the human monocytic U 937 cell line (ATCC N° CRL-1593.2) and the human HL-60 cell line (ATCC N° CCL-240), cell lines derived thereof ATCC N° CRL-2258 and CRL-2392) and normal human CD34+cells that are expanded in a culture medium comprising a cocktail of cytokine except SCF.

5. A method according to one of claims 1 to 4, wherein compounds capable of depleting specifically mast cells at a concentration below 10 μM, preferably below 1 μM are selected.

6. A method according to one of claims 1 to 5, wherein the compounds exhibiting Ratios E/S ranging from 1/1000 to 1/5 are selected.

7. A method according to one of claims 1 to 6, wherein the cell death assay further comprises a cell proliferation assay, a cell viability assay and/or an apoptosis assay.

8. A method according to one of claims 1 to 6, wherein the extent of cell death is measured by 3H thymidine incorporation, the trypan blue exclusion method, using propidium iodide or by the ⁵¹Cr-release assay.

9. A method according to one of claims 1 to 6, wherein the extent of cell death is determined by a test of intracellular esterase activity, and a test of plasma membrane integrity, preferably using fluorescent calcein and ethidium homodimer-1.

10. A method according to one of claims 1 to 6, wherein the extent of cell death is determined by discriminating between living and dead cells using DiOC₁₈ and propidium iodide.

11. A method according to one of claims 1 to 10, wherein the extent of cell death is measured by fluorometric assays of cell viability and cytotoxicity using a fluorescence microscope, a fluorometer, a fluorescence microplate reader or a flow cytometer.

12. A method according to one of claims 1 to 11, wherein the mast cells that are IL-3 dependent cells are cultured in a culture media comprising IL-3 at a concentration comprised between 0.5 and 10 ng/ml, preferably between 1 to 5 ng/ml.

13. A method according to one of claims 1 to 12, wherein compounds to be tested are selected from inhibitors of tyrosine kinases, such as Akt, c-Cbl, CRKL, Doc, p125 Fak, Fyn, Grap, Jak2, Lyn, MAPK, MATK, P13-K, PLC-γ, Raf1, Ras, SHP-1, SHP2 (Syp), Tec, Vav and Flt-3.

14. A screening method according to one of claims 1 to 12, wherein said compounds are selected from the group consisting of indolinone, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

15. A compound obtainable by the method according to one of claims 1 to 12, wherein said compound is capable of depleting mast cells and has no significant toxicity for other hematopoietic cells, preferably compounds having an E/S ratio ranging 1/1000 to 1/5.

16. Use of a compound according to claim 15 to manufacture a medicament.

17. A method for treating a disease selected from autoimmune diseases, allergic diseases, bone loss, tumor angiogenesis, inflammatory diseases, inflammatory bowel diseases (IBD), interstitial cystitis, mastocytosis, infections diseases, and CNS disorders comprising administering a compound obtainable from a method according to one of claims 1 to 14 to a mammal in need of such treatment.

18. A method for promoting hair growth and hair color revival comprising administering a compound obtainable from a method according to one of claims 1 to 14 to a human need of such treatment.

19. A method according to claim 17 for treating multiple sclerosis, psoriasis, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, Sjogren's syndrome, nodular panarteritis, autoimmune enteropathy, proliferative glomerulonephritis, active chronic hepatitis and chronic fatigue syndrome.

20. A method according to claim 17 for treating graft-versus-host disease or graft rejection in any organ transplantation including kidney, pancreas, liver, heart, lung and bone marrow.

21. A method according to claim 17 for treating subepidermal blistering disorders such as aphthous ulcers, and several bullous diseases such as pemphigus, bullous pemphigoid and cicatricial pemphigoid.

22. A method according to claim 21 comprising further administering at least one antibiotic, preferably selected from dapsone, azathioprine, erythromycin, propionylerythromycin, neomycin, gentomycin, tobramycin, and mechlocycline.

23. A method according to claim 17 for treating asthma, allergic rhinitis, allergic sinusitis, anaphylactic syndrome, urticaria, angioedema, atopic dermatitis, allergic contact dermatitis, erythema nodosum, erythema multiforme, cutaneous necrotizing venulitis, insect bite skin inflammation and blood sucking parasitic infestation.

24. A method according to claim 17 for treating skin allergic disorders such as urticaria, atopic dermatitis, allergic contact dermatitis, erythema nodosum, erythema multiforme, cutaneous necrotizing venulitis, insect bite skin inflammation and blood sucking parasitic infestation especially in dogs and cats.

25. A method according to one of claims 23 and 24 wherein the compound is administered with aerosolized formulations to target areas of a patient's respiratory tract, intranasal or topical formulation.

26. A method according to claim 17 for treating tumor angiogenesis in human.

27. A method according to claim 17 for treating skin disorders in human associated with mastocytosis, notably cutaneous mastocytosis including urticaria pigmentosa, diffuse cutaneous mastocytosis, solitary mastocytoma and bullous, erythrodermic and teleangiectatic mastocytosis.

28. A method according to claim 17 for treating category IV mastocytosis including mast cell leukemia.

29. A method according to claim 17 for treating dog mastocytoma.

30. A method according to claim 17 for treating treating inflammatory bowel diseases (IBD), such as Crohn's disease, mucositis, ulcerative colitis, and necrotizing enterocolitis.

31. A method according to claim 17 for treating interstitial cystitis in human.

32. A method according to claim 17 for treating bacterial infections in mammalian, especially in human, preferably for the treatment of recurrent bacterial infections, resurging infections after asymptomatic periods such as bacterial cystitis.

33. A method according to claim 17 for treating FimH expressing bacteria infections such as Gram-negative enterobacteria including E. coli, Klebsiella pneumoniae, Serratia marcescens, Citrobactorfreudii and Salmonella typhimurium.

34. A method according to claim 29 or 30 comprising further administering at least one antibiotic selected bacitracin, the cephalosporins, the penicillins, the aminoglycosides, the tetracyclines, the streptomycins and the macrolide antibiotics such as erythromycin; the fluoroquinolones, actinomycin, the sulfonamides and trimethoprim.

35. A method according to claim 17 for treating bone loss such as osteoporosis, including post menopausal osteoporosis, senile osteoporosis, and glucocorticoid-induced osteoporosis, osteitis fibrosa cystica, renal osteodystrophy, osteosclerosis, osteopenia, osteomalacia, fibrogenesis-imperfecta ossium, and Paget's Disease.

36. A method according to claim 17 for treating inflammatory disorders such as rheumatoid arthritis, conjunctivitis, rheumatoid spondylitis, osteoarthritis, gouty arthritis, polyarthritis, and other arthritic conditions as well as pain associated with these inflammatory diseases.
